(19) United States
(12) Patent Application Publication
(10) Pub. No.: US 2006/0165630 A1
(43) Pub. Date: Jul. 27, 2006

(54) Use of amide or ester of sugar and of fatty acid, for treating and/or preventing dry skin

(76) Inventors: Gilles Rubinstenn, PARIS (FR); Jean-Francois Michelet, Creteil (FR); Bruno Bernard, Neuilly Sur Seine (FR); Maria Dalko, Gif Sur Yvette (FR)

Correspondence Address: OLIFF & BERRIDGE, PLC, P.O. BOX 19928, ALEXANDRIA, VA, 22320, US

(21) Appl. No.: 10/527,311

(22) PCT Filed: Oct. 14, 2003
(86) PCT No.: PCT/IB03/04517

§ 371 Date: Aug. 8, 2005

(30) Foreign Application Priority Data
Oct. 15, 2002 (FR) 02 12827
Oct. 15, 2002 (FR) 02 12828

(51) Int. Cl. A61K 8/73

(52) U.S. Cl. 424/70.13

(57) ABSTRACT

The present invention relates to the use of at least one amide, a mono- or polyester of suger and of fatty acid, especially linoleic acid, for the preparation of a cosmetic of pharmaceutical composition intented for treating and/or preventing dry skin. It also relates to the use of at least one amide, a sugar mono- or polyester of fatty acid, for the preparation of a cosmetic composition intented for treating oligoseborrhoeic dry skin and/or for stimulating sebum production. It also relates to the use of at least one amide, a mono- or polyester of sugar of linoleic acid for the preparation of a cosmetic or pharmaceutical composition, intented for generating 13-hydroxy-octadecadienoic acid and/or for treating and/or preventing skin disorders and/or disorders of the pilosebaceous unit associated with a deficit of linoleic acid.

The present invention relates to the use of at least one amide or a mono- or polyester of sugar of fatty acid for the preparation of a therapeutic or cosmetic composition intended for preventing and/or treating dry skin and especially for treating oligoseborrhoeic dry skin as well as the use of at least one amide or a mono- or polyester of sugar and of linoleic acid for the preparation of a therapeutic or cosmetic composition intended for treating and/or preventing disorders associated with dryness of the skin due in particular to a deficiency of linoleic acid.

It will be recalled that the skin is made up of three superposed layers, from the surface into the body: the epidermis, the dermis and the hypodermis (or subcutaneous tissue).

The epidermis, the outermost layer of the skin, is a keratinized stratified pavement epithelium, the constitution of which includes four different cellular populations: keratinocytes, melanocytes, Langerhans' cells and Merkel cells. The epidermis contains neither blood nor lymphatic vessels, but it does contain numerous free nerve endings.

The keratinocytes are constantly undergoing morphological development testifying to their keratinization underlying the role of protective barrier (mechanical and chemical) of the epidermis.

This development is in the direction from the deeper layers towards the surface and a cross-section through the epidermis reveals four superposed layers from deep down towards the surface: the basal layer or stratum germinativum, the spinous layer or stratum spinosum, the granular layer or stratum granulosum and the horny layer or stratum corneum (compact, then desquamating).

The dermis, underneath the epidermis, nourishes and supports the latter. It is formed from a dense network of interwoven fibres: on the one hand, collagen fibres, which gives the dermis its resistance to forces of compression, and elastic fibres on the other hand, which give the skin its elasticity.

The hypodermis is essentially a bed of fat.

The skin also contains ancillary structures, in particular the sebaceous glands. These glands secrete an oily substance called sebum, which forms an impermeable film on the surface of the epidermis; they are located near the hair follicles, forming the pilosebaceous unit. Together with sweat, produced by the eccrine or apocrine glands, the sebum constitutes a natural moisturizer of the epidermis and helps to increase its elasticity and strength.

In addition, it constitutes the route for natural excretion of endogenous vitamin E, a potent antioxidant that helps to protect the surface layers of the epidermis against injury, especially that caused by UV.

Sebum consists essentially of a more or less complex mixture of lipids. Classically, the sebaceous gland produces squalene, triglycerides, aliphatic waxes, cholesterol waxes and, possibly, free cholesterol. It is the action of bacterial lipases that converts a variable proportion of the triglycerides that form into free fatty acids.

The cell in the sebaceous gland responsible for the expression of sebum is the sebocyte. In fact, sebum production is associated with a programme of terminal differentiation of this cell. During this differentiation, the metabolic activity of the sebocyte is essentially focused on lipid biosynthesis and more precisely on the neosynthesis of fatty acids.

The density of sebaceous glands is not identical over the whole surface of the skin: some regions of the skin have a very high density of sebaceous glands, whereas in other regions their density is much lower or they are even absent.

In general, dry skin and especially oligoseborrhoeic skin is characterized by insufficient secretion and excretion of sebum. Classically, a sebum level below 100 μg/cm², measured in the T zone of the face, by the method described in FR 2 368 708, can be regarded as typical of dry skin.

Dry skin may be due to an endogenous insufficiency of sebum production. An example of dry skin, or it becoming so, is observed as the skin ages. Furthermore, insufficient production of sebum may be caused, in particular, by certain pharmaceutical treatments, such as those involving corticoids.

Dry skin is often associated with a defect of desquamation, a sallow complexion and/or an atonic skin texture. Micro-inflammatory manifestations of the dermatitis type, for example, may often appear on this type of skin. Moreover, a dry scalp is often associated with dull, lifeless hair.

Consequently, a compound that can stimulate the production of lipids, of which the sebum is composed, by the cells of the sebaceous gland would definitely be of interest for the treatment of disorders associated with dry, oligoseborrhoeic skin.

Certain steroidal hormones or pre-hormones of the DHEA type are already known to exert an activating effect on sebaceous function. In particular, they have already been proposed as an agent for restoring normal sebaceous function when it has deteriorated through age.

However, the use of DHEA, as with all derivatives that can lead metabolically to a sex hormone, raises additional problems connected with safety of use. In fact, it is not possible to exclude secondary effects connected with the use of this type of hormone, such as masculinization in women, liver damage and increased risk of prostate cancer in men or of breast cancer in women.

Therefore a particular aim of the present invention is to propose compounds that can advantageously replace the activators of sebaceous function used up to now.

Unexpectedly, the inventors found that the amides, sugar monoesters and polyesters of fatty acid exhibited significant activity in respect of oligoseborrhoea. It appears that the amides and esters of sugar and of fatty acid according to the invention stimulate sebum production.

Accordingly, the compositions according to the present invention are of particular interest for the treatment of dry skin and especially oligoseborrhoeic skin.

The skin acts essentially as a barrier to the external environment that results from a complex, multifactorial organization.

However, this function is based in particular on the quality of the epidermis, which depends notably on the balance between proliferation and differentiation of the keratinocytes of the epidermis.

There are numerous cosmetic or dermatologic actives that aim to guarantee or re-establish skin balance. These actives protect, nourish, moisturize and calm the skin, or they regulate intercellular communication.

Disturbance of skin balance can be manifested in various ways. In particular, it can lead to the triggering of inflammatory processes, disturbance of sebaceous function, hyperkeratinization, as well as an increase in the barely perceptible loss of water and more generally to dryness of the skin. These events have an adverse effect on skin comfort and/or aesthetics. In addition, they are likely to affect the state of health of the epidermis or its appendages by altering their flora, for example by promoting their colonization by various microorganisms.

It has also been known for many years that a diet deficient in vitamin F and more particularly one of its essential components, namely linoleic acid, affects the skin balance. This imbalance is reflected notably in dryness of the skin and especially in an elevated imperceptible water loss, as well as altered cutaneous desquamation. Dermatitis, skin redness, formation of sores and impairment of the healing process have also been observed. It can also be reflected in depigmentation, and loss of hair, eyebrows and/or body hair.

It is now known that in the cutaneous epidermis, linoleic acid is converted by 15-lipoxygenase in the epidermis, mainly to β-hydroxy-octadecadienoic acid (also known by its abbreviation 13-HODE) which moderates tissue proliferation either directly or indirectly.

It is also known that linoleic acid deficiency leads to a deficiency of 13-HODE.

Finally, it has been reported that topical applications of linoleic acid on skin that is deficient in linoleic acid made it possible to restore the imperceptible water loss to a normal level. Furthermore, it has been demonstrated in an animal model that hyperproliferation of epidermal keratinocytes, linked to deficiency of essential fatty acids, can be reversed by topical application of 13-HODE (Miller et al., 1990, J. Invest. Dermatol., 94, 353-358).

However, although the experimental use of 13-HODE led to positive results being obtained, its wide-scale use can scarcely be envisaged, as it is not a readily available molecule, in contrast to linoleic acid, which is present in several natural oils. What is more, both 13-HODE and linoleic acid, as well as its commonest form, namely vitamin F, in which it is present in a high proportion, are, owing to their chemical nature, unstable in the air and undergo peroxidation.

Therefore a particular aim of the present invention is to propose compounds that are more resistant to peroxidation in the air than linoleic acid and are suitable for treating and/or preventing dryness of the skin, notably because they are able to generate 13-HODE.

After extensive research, the applicant has now demonstrated that the esters or amides of linoleic acid and of sugar have remarkable properties, justifying their use for improving the condition of the epidermis and/or of the pilosebaceous unit, and especially for treating and/or preventing dry skin.

In particular they can improve the condition of the epidermis on the entire skin surface of an individual, including areas of the skin with few if any sebaceous glands, such as the palms, the medial surface of the arms and the medial surface of the legs.

The product used in the present invention has several advantages, for instance it contains an essential fatty acid that is naturally present in the human body.

Furthermore, it is very well tolerated by the skin.

This product is significantly more resistant to peroxidation in the air than the products used in the prior art for similar indications, and in particular is significantly more stable than linoleic acid.

Finally, it can be synthesized easily, on an industrial scale, at relatively low cost.

A first aspect of the present invention relates to the use of at least one amide or one sugar mono- or polyester of fatty acid and in particular of linoleic acid, as active principle, for the preparation of a cosmetic or pharmaceutical composition intended for preventing or treating dry skin.

According to another of its aspects, the present invention also relates to the use of at least one amide or one sugar mono- or polyester of fatty acid, as active principle, for the preparation of a cosmetic or pharmaceutical composition intended for the treatment of dry, oligoseborrhoeic skin.

According to another of its aspects, the present invention further relates to the use of at least one amide or one sugar mono- or polyester of fatty acid, as active principle, for the preparation of a cosmetic or pharmaceutical composition intended for stimulating sebum production.

According to another of its aspects, the present invention also relates to the use of at least one amide or one mono- or polyester of sugar of linoleic acid, as active principle, for the preparation of a cosmetic or pharmaceutical composition intended for generating 13-HODE in the cutaneous epidermis.

Another object of the invention is the use of at least one amide or one mono- or polyester of sugar of linoleic acid, as active principle, for the preparation of a cosmetic or pharmaceutical composition intended for treating and/or preventing skin disorders and/or disorders of the pilosebaceous unit associated with a deficiency of linoleic acid.

### GENERAL DESCRIPTION

Sugar is the generic name commonly used for designating substances that possess several alcohol functions, with or without aldehyde or ketone functions, and with at least C₃.

More precisely, this term covers the oses, also called monosaccharides, which contain from three to nine carbon atoms, the oligosaccharides resulting from the condensation of a small number of oses, generally less than 5, by means of glycosidic bonds, like the disaccharide, and the polysaccharides in which a larger number of oses are joined together.

Within the scope of the present invention, the sugar in question is more particularly a mono- or oligosaccharide and especially a mono- or disaccharide.

By way of illustration, it will be recalled that the monosaccharides are either aldoses or ketoses which, classically, are represented respectively in a linear form by one of the following formulae: 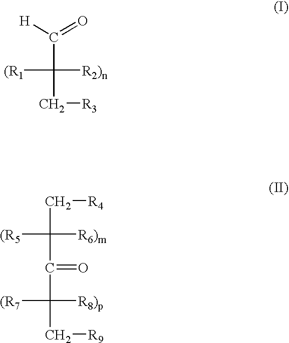
in which n represents an integer equal to or greater than 1, m and p represent, independently of one another, an integer equal to or greater than 1 and R₁, R₂, R₃, R₄, R₅, R₆, R₇, R₈ and R₉ represent, independently, a hydrogen atom, a hydroxyl group, an amine function or an N-acetylamide function.

In the case of the present invention, such a sugar is functionalized on at least one of the hydroxyl or amine functions represented by R₁, R₂, R₃, R₄, R₅, R₆, R₇, R₈, R₉ with a fatty acid.

The present invention covers mixtures, racemic or not, of isomers of L and D configuration of these sugars as well as their L and D isomers in pure form.

Among these mono- and disaccharides, those derived from pentoses and/or hexoses are quite particularly suitable.

The D series isomers of mono- and disaccharides, especially of the pentose or hexose type, can be used more particularly according to the invention.

Generally, the predominant form of the hexoses and pentoses is a cyclized form, obtained, starting from one of the aforementioned linear forms, by spontaneous reaction of a carbonyl function, in particular aldehyde, with an alcohol function so as to form a hemiacetal. This cyclization leads to the formation of the sugars in the corresponding pyranose and furanose form. The present invention also covers these cyclized forms, called furanic in the case of a pentose and pyranic in the case of a hexose as well as the corresponding alpha and beta isomers, in pure form or as a mixture.

As a non-limiting illustration of the mono- and disaccharides that can be used according to the invention, we may mention more particularly talose, fucose, ribose, idose, arabinose, gulose, xylose, lyxose, altrose, allose, glucose, mannose, galactose, lactose, sucrose, trehalose, cellobiose, maltose, fucose alpha 1-3 glucose, fructose and their derivatives. We may mention in particular glucosamine, fructosamine, galactosamine, fucose alpha 1-4 glucosamine and their derivatives, especially N-acetylated derivatives, as being representative of the mono- and disaccharides possessing an unsubstituted amine function.

Maltose, sucrose, cellobiose, trehalose, lactose, fucose alpha 1-3 glucose, and fucose alpha 1-4 glucosamine are quite especially suitable as disaccharides for the invention.

Monosaccharides of the pentose series, for example lyxose, xylose, arabinose and ribose, and of the hexose series such as talose, fucose, galactose, idose, gulose, mannose, glucose, altrose, allose, glucosamine, galactosamine, N-acetyl glucosamine, N-acetyl galactosamine and fructose, are also suitable for the invention.

The mixture of the alpha D- or beta D-isomers of glucose is used more particularly within the scope of the present invention.

The sugars are combined in an amidified or esterified form and more particularly esterified with the fatty acid in question.

The mono- or disaccharide is in particular esterified with the fatty acid in question on a hydroxyl function.

In this case, the mono- or polysaccharide and especially the mono- or disaccharide can be mono- or polyesterified and the esterification positions can be located at positions 1, 2, 3, 4 and/or 6, especially at positions 1, 2, 3 and/or 6 and in particular at positions 1, 3 and/or 6, and more particularly at position 6.

In the special case when the derivative of fatty acid according to the invention is an amide, this amidation is located at position 2.

The fatty acids considered according to the invention are more particularly long-chain fatty acids, i.e. they can contain more than 14 carbon atoms.

Their hydrocarbon chain can be saturated or contain one or more double bonds. We may mention in particular the saturated fatty acids such as palmitic (C₁₆), stearic (C₁₈), arachidic (C₂₀), behenic (C₂₂) and lignoceric (C₂₄) acids and the unsaturated fatty acids such as palmitoleic (C₁₆), oleic (C₁₈), linoleic (C₁₈), linolenic especially in its a and γ forms (C₁₈) and arachidonic (C₂₀) acids, as representative of these fatty acids.

Among these fatty acids, linoleic acid and stearic acid, and more particularly linoleic acid, are of quite especial interest.

These acids can react in a pure form with the sugar in question, or in the form of one of their mixtures, natural or synthetic. In this case, linoleic acid can be employed in the form of vitamin F, which is a natural mixture of linoleic acid notably with minor amounts of oleic and stearic acids.

According to a particular variant of the invention, the composition contains at least one sugar monoester of linoleic acid.

The sugar ester of linoleic acid used can in particular be derived from glucose, notably the monoester at position 1, 3 or 6 of glucose, especially of α D- or β D-glucose, of linoleic acid, and more particularly of the ester at position 6.

In particular, the compound used is a 6-O-octadeca-9,12-dienoyl-D-glucopyranose.

The ester at position 3 of glucose, especially of αD- or βD-glucose, of stearic acid and the amide at position 2 of glucosamine of linoleic acid are also of particular interest for the stimulation of sebum production.

The esters at position 1 and 6 of glucose, especially of αD- or βD-glucose of linoleic acid are of particular interest for generating 13-HODE.

According to a particular embodiment, the invention relates to the use of an amide or of a sugar mono- or polyester of linoleic acid as defined previously and in particular of 6-O-octadeca-9,12-dienoyl-D-glucopyranose, used in the form of a mixture comprising at least two different compounds. In particular this mixture can contain at least one amide or one sugar mono- or polyester of linoleic acid and one other amide or one other sugar mono- or polyester of fatty acid in particular as defined previously.

More particularly, the present invention relates to the use of a monoester of linoleic acid and of glucose and in particular 6-O-octadeca-9,12-dienoyl-D-glucopyranose, employed in the form of at least two compounds that can be represented respectively by the following formula (III):

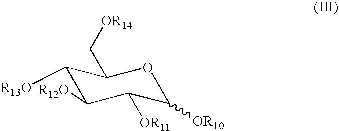

in which:

R₁₀, R₁₁, R₁₂, R₁₃ and R₁₄ represent, independently, a hydrogen atom or an OC—R radical, with R representing a linear, saturated or unsaturated hydrocarbon chain containing from 11 to 21 carbon atoms and

with at least one of the compounds having, as at least one of the radicals R₁₀ to R₁₄, the linoleoyl radical.

The ratio between the number of ester functions of the compound of formula (III) and the number of initial hydroxyl functions, or degree of esterification, for a glucose molecule, varies from 0.2 to 1. Notably, it is less than or equal to 0.6, and in particular less than or equal to 0.4.

In formula (III) defined above, the radical R can in particular represent a linoleyl, oleyl, palmityl, stearyl, lauryl, myristyl, arachidyl, behenyl, lauroleyl, myristoleyl, palmitoleyl and/or linolenyl radical especially in their a or γ forms.

In particular, the mixture can contain, in addition to an ester, notably a monoester, of glucose of linoleic acid, an ester, notably monoester, of glucose of oleic acid and/or an ester, notably monoester, of glucose of stearic acid.

With regard to the preferred sites of esterification, they correspond to those mentioned previously.

According to a particular embodiment, 50 to 100% of the glucose esters in the mixture are esterified at position 6 of the glucose, notably at least 55%, in particular at least 80%, and more particularly at least 90%.

In particular, the mixture used according to the invention can contain in addition to an ester, notably monoester, of linoleic acid of glucose, at least one ester, notably monoester, of oleic acid and of glucose; at least one ester, notably monoester, of fatty acid and of glucose, the said fatty acid being selected from palmitic and stearic acid; at least one ester, notably monoester, of fatty acid and of glucose, the said fatty acid being selected from lauric, myristic, arachidic, behenic, lauroleic, myristoleic, palmitoleic and linolenic acid.

When the 6-O-octadeca-9,12-dienoyl-D-glucopyranose as defined previously is used in the form of a mixture as defined previously, the total proportion by weight of ester of linoleic acid and of glucose relative to the total weight of the said mixture is generally from 40 to 90%, notably it is greater than or equal to 50%, in particular greater than or equal to 60%, and more particularly less than or equal to 80%, notably less than 75%, and in particular varies from 68 to 72%.

Generally, the proportion of 6-O-octadeca-9,12-dienoyl-D-glucopyranose relative to the total weight of the said mixture is greater than or equal to 40%, notably greater than or equal to 50% and in particular varies from 60 to 80%.

When the mixture as defined previously contains at least one ester of oleic acid and of glucose, this is generally present in a proportion by weight relative to the total weight of the said mixture of 5 to 20%, notably greater than or equal to 8%, in particular greater than or equal to 10%, more particularly greater than or equal to 12%, and in particular less than or equal to 17% and notably in a proportion varying from 14 to 15% by weight.

When the mixture as defined previously contains at least one ester of palmitic acid and of glucose, this is generally present in a proportion by weight relative to the total weight of the said mixture of 2 to 20%, notably greater than or equal to 5%, in particular greater than or equal to 7%, and notably less than or equal to 15% and in particular in a proportion varying from 9 to 12%.

When the mixture as defined previously contains at least one ester of stearic acid and of glucose, this is generally present in a proportion by weight relative to the total weight of the said mixture from 0.1 to 7%, notably greater than or equal to 0.5%, in particular greater than or equal to 1%, and notably less than or equal to 5%, in particular in a proportion varying from 2 to 4% by weight.

When the mixture as defined previously contains at least one ester of fatty acid and of glucose, the said fatty acid being selected from lauric, myristic, arachidic, behenic, lauroleic, myristoleic, palmitoleic and linolenic acid, the said ester or the set of the said esters is generally present in a proportion by weight relative to the total of the said mixture less than or equal to 10%, notably from 0.1 to 4%, and in particular from 0.15 to 2%.

According to a particular embodiment of the invention, the esters mentioned previously are monoesters.

The mixture as defined previously can in addition contain at least one diester of glucose and of one fatty acid or of two different fatty acids, notably selected from linoleic, oleic, palmitic, stearic, lauric, myristic, arachidic, behenic, lauroleic, myristoleic, palmitoleic and linolenic acids.

In such an embodiment, the said diester or the set of the said diesters is generally present in a proportion by weight relative to the total weight of the said mixture less than or equal to 10%, notably varying from 0.1 to 4%, and in particular from 0.15 to 2% by weight.

Thus, the mixture that can be used in the invention generally contains, irrespective of positions:

from 40 to 80 wt. %, preferably 60 to 75 wt. %, preferentially 68-72 wt. %, of monoester of glucose and of linoleic acid,

from 10 to 20 wt. %, preferably 12 to 17 wt. %, preferentially 14-15 wt. %, of monoester of glucose and of oleic acid,

from 5 to 20 wt. %, preferably 7 to 15 wt. %, preferentially 9-12 wt. %, of monoester of glucose and of palmitic acid,

from 0.5 to 7 wt. %, preferably 1 to 5 wt. %, preferentially 2-4 wt. %, of monoester of glucose and of stearic acid,

from 0 to 10 wt. %, notably 0.10-4 wt. %, or even 0.15-2 wt. %, of one or more monoesters of glucose and of lauric, myristic, arachidic, behenic, lauroleic, myristoleic, palmitoleic and/or linolenic acid,

from 0 to 10 wt. %, notably 0.10-4 wt. %, or even 0.15-2 wt. %, of diesters of glucose and of one or more acids selected from lauric, myristic, arachidic, behenic, lauroleic, myristoleic, palmitoleic, linoleic, oleic, palmitic, stearic and/or linolenic acids.

In particular, the mixture can contain:

from 40 to 80 wt. %, preferably 60 to 75 wt. %, preferentially 68-72 wt. %, of ester of glucose and of linoleic acid and principally 6-O-octadeca-9,12-dienoyl-D-glucopyranose, 1-O-octadeca-9,12-dienoyl-D-glucopyranose, 2-O-octadeca-9,12-dienoyl-D-glucopyranose and/or 3-O-octadeca-9,12-dienoyl-D-glucopyranose,

from 10 to 20 wt. %, preferably 12 to 17 wt. %, preferentially 14-15 wt. %, of ester of glucose and of oleic acid, and principally 6-O-octadeca-9-enoyl-D-glucopyranose, 3-O-octadeca-9-enoyl-D-glucopyranose, 1-O-octadeca-9-enoyl-D-glucopyranose and/or 2-O-octadeca-9-enoyl-D-glucopyranose,

from 5 to 20 wt. %, preferably 7 to 15 wt. %, preferentially 9-12 wt. %, of ester of glucose and of palmitic acid, and principally 6-O-hexadecanoyl-D-glucopyranose, 3-O-hexadecanoyl-D-glucopyranose, 1-O-hexadecanoyl-D-glucopyranose and/or 2-O-hexadecanoyl-D-glucopyranose,

from 0.5 to 7 wt. %, preferably 1 to 5 wt. %, preferentially 2-4 wt. %, of ester of glucose and of stearic acid, and principally 6-O-octadecanoyl-D-glucopyranose, 3-O-octadecanoyl-D-glucopyranose, 1-O-octadecanoyl-D-glucopyranose and/or 2-O-octadecanoyl-D-glucopyranose,

from 0 to 10 wt. %, notably 0.10-4 wt. %, or even 0.15-2 wt. %, of one or more esters of glucose and of lauric, myristic, arachidic, behenic, lauroleic, myristoleic, palmitoleic and/or linolenic acid,

from 0 to 10 wt. %, notably 0.10-4 wt. %, or even 0.15-2 wt. %, of diesters of glucose and of one or more acids selected from lauric, myristic, arachidic, behenic, lauroleic, myristoleic, palmitoleic, linoleic, oleic, palmitic, stearic and/or linolenic acids.

According to a particular embodiment of the invention, the mixture used can be obtained by esterification of D-glucose by vitamin F.

It is known that vitamin F, a compound that occurs naturally in fats and notably in linseed oil, sunflower oil and safflower oil, consists of a mixture of fatty acids, mainly from C₁₂ to C₂₀.

Thus, it is considered that vitamin F generally comprises (wt. %):

from 75 to 80 wt. % of linoleic acid,

from 10 to 15 wt. % of oleic acid,

from 4 to 8 wt. % of palmitic acid,

from 0.5 to 3 wt. % of stearic acid, and

from 0 to 10 wt. % of one or more other acids such as lauric, myristic, arachidic, behenic, lauroleic, myristoleic, palmitoleic and linolenic acids.

The product obtained by esterification by vitamin F therefore generally consists of a mixture of various esters, resulting in particular from the presence of the various acids that make up vitamin F.

In particular, the reaction of esterification can be carried out according to all known methods. Synthesis can in particular be effected starting from the chloride of linoleic acid or from the chloride of vitamin F and of D-glucose, in accordance with the method described by Reinfeld et al., in “Die Stärke”, No. 6, pages 181-189, 1968. In particular, a more detailed account of this method is given in patent EP 485 25 1.

The sugar esters or amides of linoleic acid can be prepared in accordance with conventional methods.

In general, the compositions according to the invention are of particular interest for physiologically restoring a suitable state of hydration for the skin barrier.

Thus, the dryness that can be treated according to the invention can be an acquired, transient dryness, i.e. dryness associated with dehydration of the skin caused for example by cold, heat, detergents and/or hard water. It might also be an acquired, permanent dryness such as that due to chronological aging of the skin generally associated with a loss of functionality of the sebaceous glands and hence with some degree of sebum deficiency. Finally, the dryness may be constitutional, i.e. manifested chronically by the patient or it may be of genetic origin, like ichthyosis.

Insofar as the inventors detected a stimulating action of the sugar esters or amides of fatty acid and notably of the glucose esters of fatty acid on sebum production, the compositions according to the invention prove particularly advantageous for treating disorders associated with oligoseborrhoea.

Accordingly, the compositions according to the invention can be used effectively for treating skin displaying insufficient secretion or excretion of sebum, as well as the disorders generally associated with this type of dryness, for example a disturbance of desquamation and/or micro-inflammatory symptoms of the dermatitis type.

According to a variant of the invention, the sugar amide or mono- or polyester of linoleic acid as defined previously and in particular 6-O-octadeca-9,12-dienoyl-D-glucopyranose or the mixture as defined previously can be used in the treatment and/or the prevention of excessive cutaneous desquamation, dryness of the skin, in particular associated with an abnormally high level of imperceptible water loss, and dermatitis. The amide or the sugar mono- or polyester of linoleic acid as defined previously and in particular 6-O-octadeca-9,12-dienoyl-D-glucopyranose or the mixture as defined previously can also be used for the treatment and/or the prevention of disorders of cicatrization, redness and irritation. The amide or the sugar mono- or polyester of linoleic acid as defined previously and in particular 6-O-octadeca-9,12-dienoyl-D-glucopyranose or the mixture as defined previously can also improve the state of health of the epidermis and in particular prevent its colonization by microorganisms, by improving the condition of the skin barrier. Moreover, they can also improve, or even re-establish, the differentiation/proliferation balance of the keratinocytes. Furthermore, they can be used advantageously for the treatment and/or the prevention of hyperkeratosis of the infundibular epithelium.

In the compositions, the sugar ester(s) or amide(s) of fatty acid can be present in proportions ranging from 0.001 to 30 wt. % relative to the total weight of the composition, and in particular from 0.01 to 15 wt. %, and notably from 0.1 to 5 wt. %, for example greater than or equal to 0.5 wt. %.

The amount of the sugar ester(s) or amide(s) of linoleic acid can easily be determined by a person skilled in the art, notably according to the nature of the composition and/or the desired effect.

Generally speaking, in the compositions, the sugar ester(s) or amide(s) of linoleic acid can be present in proportions varying from 0.001 to 30 wt. % relative to the total weight of the composition, in particular less than or equal to 20 wt. %, more particularly from 0.01 to 15 wt. %, notably from 0.1 to 5 wt. %, and for example greater than or equal to 0.5 wt. %.

In particular, the compositions contain from 0.1 to 5% of 6-O-octadeca-9,12-dienoyl-D-glucopyranose.

In the compositions according to the invention, the active principle in the form of a compound or a mixture, can additionally be combined with an effective quantity of at least one other active agent, i.e. a compound that is known to exert a therapeutic or beneficial action on the skin despite the undesirable effects possibly associated with this additional compound.

For example, this known compound may produce an undesirable effect such as the development of dry skin notably by limiting the production of sebum. As examples of such compounds we may mention the corticoids, in particular cortisone, hydrocortisone and betamethasone; indometacin; derivatives of retinoic acid.

As compounds suitable for combining with the esters and amides according to the invention, consideration may be given in particular to compounds that are already known to display a moisturizing action.

The term “moisturizer” means:

either a compound that acts on the barrier function, with a view to maintaining the hydration of the stratum corneum, or an occlusive compound. By way of illustration and without limitation we may mention ceramides, sphingoid base compounds, lecithins, glycosphingolipids, phospholipids, cholesterol and its derivatives, phytosterols (stigmasterol, β-sitosterol, campesterol), essential fatty acids, 1,2-diacylglycerol, 4-chromanone, the pentacyclic triterpenes such as ursolic acid, vaseline and lanolin;

or a compound that increases the water content of the stratum corneum directly, such as threalose and its derivatives, hyaluronic acid and its derivatives, glycerol, pentanediol, sodium pidolate, serine, xylitol, sodium lactate, glycerol polyacrylate, ectoin and its derivatives, chitosan, oligo- and polysaccharides, cyclic carbonates, N-lauroyl pyrrolidone carboxylic acid, and N-α-benzoyl-L-arginine;

or a compound that activates the sebaceous glands such as vitamin D and its derivatives.

The composition can also contain one or more agents that stimulate the proliferation and/or differentiation of the keratinocytes.

The agents that stimulate the proliferation of keratinocytes, that can be used in the composition according to the invention, notably include the retinoids such as retinol and its esters, including retinyl palmitate; phloroglucinol; the nut cake extracts marketed by the company GATTEFOSSE; the Solanum tuberosum extracts marketed by the company SEDERMA.

The agents that stimulate differentiation of the keratinocytes, that can be used in the composition according to the invention, notably include minerals such as calcium; the lupin extract marketed by the company SILAB with the trade name Photopreventine®; sodium beta-sitosteryl sulphate marketed by the company SEPORGA with the trade name Phytocohesine®; the maize extract marketed by the company SOLABIA with the trade name Phytovityl®.

One or more anti-inflammatory and calming agent(s) can also be combined with the active principles according to the invention.

“Anti-inflammatory agent” means any compound that is capable of inhibiting the principal enzymes involved in the inflammatory process (arachidonic acid cascade), namely: phospholipases A2 (PLA2); lipoxygenases (Lox); human prostaglandin synthases.

“Calming agent” means in particular the antagonists of substance P, the CGRP antagonists and the bradykinin antagonists.

Among the substances that are effective as anti-inflammatory agents, the following agents may be mentioned, non-limitatively: the pentacyclic triterpenes, such as β-glycyrrhetinic, ursolic, oleanolic, and betulinic acids, their salts and derivatives; extracts of Paeonia suffruticosa and/or lactiflora, of Rosmarinus officinalis, of willowherb, of Pygeum, of Boswellia serrata, of Centipeda cunnighami, of Helianthus annuus, of Cola nitida, of clove and of Bacopa moniera; the salts of salicylic acid and in particular zinc salicylate; aspirin; ibuprofen; extracts of algae, in particular of Laminaria saccharina; canola oil, Tamanu oil, calophyllum oil, omega-3 unsaturated oils such as the oils from muscat rose, from cassis, from ecchium, from fish; α-bisabolol and camomile extracts; allantoin; the phosphoric diester from vitamin E and C; capryloyl glycine; the tocotrienols; piperonal; aloe vera; the phytosterols.

Examples of antagonists of substances P are in particular: strontium salts; water from hot springs; bacterial extracts and in particular the extract from non-photosynthetic filamentous bacteria prepared from bacteria of the order Beggiatoales, and more especially of the genus Vitreoscilla.

The composition can also contain one or more antibacterial agent(s) including for example triclosan, phenoxyethanol, octoxyglycerol, octanoylglycine, 10-hydroxy-2-decanoic acid, caprylyl glycol, farnesol and azelaic acid.

The composition can additionally contain at least one active agent such as a calcium antagonist or a free radical trapping agent.

The composition according to the invention can additionally contain as active agent at least one organic filter active in the UV-A and/or UV-B. By way of non-limiting illustration of these filters, we may in particular mention those stated below, by their CTFA name: the derivatives of para-aminobenzoic acid, the derivatives of dibenzoylmethane, the cinnamic derivatives, the derivatives of β,β′-diphenylacrylate, the derivatives of benzophenone, the derivatives of benzylidene camphor, the derivatives of phenyl benzimidazole, the derivatives of triazine, the derivatives of phenyl benzotriazole, the anthranilic derivatives, the derivatives of imidazolines and the derivatives of benzalmalonate. The inorganic filters that can be used in the composition according to the invention can be nanopigments of metal oxides, coated or uncoated, for example nanopigments of titanium oxide, iron oxide, zinc oxide, zirconium oxide or cerium oxide.

The medium used in these compositions can consist of water or a mixture of water and a solvent or a mixture of solvents, the solvents being selected from the organic solvents that are acceptable cosmetically or pharmaceutically and more particularly from the C₁-C₄ lower alcohols, the alkyleneglycols; the alkyl ethers of alkyleneglycol and of dialkyleneglycol. The solvents, when present, can be present in proportions ranging from 5 to 95 wt. % relative to the total weight of the composition.

The compositions according to the invention containing these compounds can be in the form of lotions, emulsions, creams, gels, and can if necessary be pressurized in an aerosol.

The composition used within the scope of the present invention is generally applied topically. Consequently, it is preferably formulated in a form appropriate to this type of application. In particular it can be a liquid, a semi-solid or a solid preparation such as an ointment, a lotion, a gel, a cream or an emulsion.

According to a particular embodiment of the invention, the composition is formulated as an oil-in-water emulsion. This type of formulation is advantageous in that the oily phase of the said emulsion, mimics in its constituents the composition of sebum and therefore imparts better availability of the active principle especially with respect to the sebaceous gland. The oily component of this emulsion can be natural or synthetic, and is of course suitably safe.

These compositions can of course contain other adjuvants that are usually employed in the cosmetic or pharmaceutical field, for producing topical compositions, such as surfactants, thickening agents, cosmetic agents such as, by way of non-limiting examples, polymers, proteins and more especially synthetic oils, preservatives, alkalizing or acidifying agents. The pH of these compositions can vary from 3 to 9 and preferably from 5 to 8.

The thickening or gelling agents can be selected from the biopolysaccharides, such as xanthan gums and scleroglucans, cellulose derivatives such as hydroxypropylcellulose and methylcellulose, polyacrylic acids crosslinked or not, polyethyleneglycols and their derivatives and combinations of anionic polymers and cationic polymers, such as those described in French patent No. 2 598 611.

The thickening agents can be present in proportions ranging from 0.1 to 5 wt. %, and in particular from 0.4 to 3 wt. % relative to the total weight of the composition.

The synthetic oils can be selected from the paraffins and the polydecenes.

The present invention also relates to a method of cosmetic treatment of the skin, characterized in that at least one composition as defined above is applied to the area to be treated.

Application is more particularly carried out by topical application.

The frequency and the duration of the application, as well as the quantity of the composition according to the invention applied onto the skin can easily be determined by a person skilled in the art, notably according to the nature of the composition and/or the desired effect.

Typically, the composition is applied once, twice, three times, until six times a day, during one day to several months by deposition of a thin layer on the skin area to be treated.

The invention is illustrated in greater detail in the following examples.

In these examples, the compound 6-O-octadeca-9,12-dienoyl-D-glucopyranose is described in the literature.

### DIAGRAM

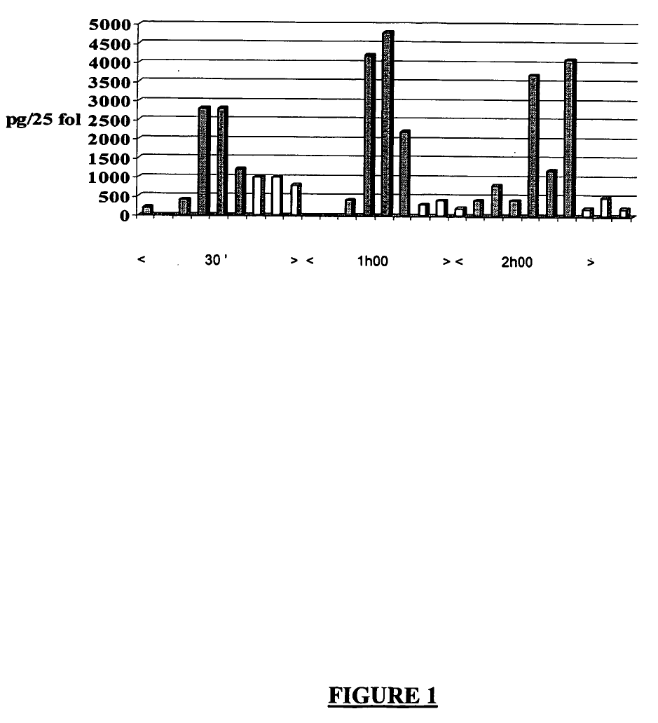

FIG. 1: Histogram representing the synthesis of 13-HODE by the hair follicles surviving in culture measured in accordance with example 9.

### EXAMPLE 1

### Preparation of the Glucose Ester of Vitamin F (Mostly Ester at Position 6)

In a 500-ml three-necked flask, dilute 17 ml of pivaloyl chloride in 100 ml of tetrahydrofuran; add, under inert atmosphere and at 0° C., a mixture of 37.3 g of vitamin F and 19.3 ml of triethylamine previously dissolved in 100 ml of tetrahydrofuran; stir for one hour then filter the salts formed to obtain a solution.

In a 2-litre three-necked flask, dissolve 96 g of D-glucose in 1.15 litres of pyridine, then add the aforementioned solution, under inert atmosphere, at room temperature. Stir the mixture overnight.

Evaporate the reaction medium to dryness, under vacuum to eliminate the pyridine, then extract the paste obtained (with water/organic solvent), and dry, filter and evaporate the organic phase.

49 g of a yellow paste of ester of vitamin F is obtained (yield: 83%).

¹H NMR spectrum (DMSO) 200 MHz: δ (ppm): 0.85; 1.23; 1.50; 2.00; 2.26; 2.73; 3.03; 3.13; 3.40; 3.76; 3.97; 4.25; 4.53; 4.76; 4.89; 5.04; 5.32; 6.34.

¹³C NMR spectrum (DMSO) 200 MHz: δ (ppm): 13.95; 22.12; 24.48; 25.23; 26.62; 28.46 to 29.08; 31.32; 33.44; 63.91; 69.14; 70.57; 72.19; 72.86; 92.30; 127.77; 129.73; 172.92.

The ¹H and ¹³C NMR spectra (DMSO) 200 MHz correspond to the expected structure.

### EXAMPLE 2

### Preparation of the Glucose Ester of Vitamin F (Mostly Ester at Position 3)

Place 20 g of vitamin F dissolved in 300 ml of anhydrous toluene in a 500-ml flask, under a nitrogen atmosphere, and add three drops of DMF to catalyse the reaction. Then add 12.6 ml of oxalyl chloride dropwise (release of gas) and stir for three hours at 25° C. Concentrate the reaction medium to the maximum, then dilute in 200 ml of dichloromethane. The chloride of vitamin F to be used in the next step is obtained.

Place 29.6 g of diacetone-D-glucose dissolved in 200 ml of dichloromethane, and 26 ml of triethylamine, in a 500-ml three-necked flask fitted with a condenser and a dropping funnel, under a nitrogen atmosphere.

Maintain the temperature at about 10° C. with an ice water bath.

Add, dropwise, 200 ml of the chloride of vitamin F obtained previously, while maintaining the temperature at about 10° C. Then stir the reaction medium for 2 hours at room temperature.

Dilute the pasty mixture obtained by adding 200 ml of dichloromethane. Then wash several times: (i) addition of distilled water and removal of the upper, aqueous solution, (ii) addition of a solution of 1N hydrochloric acid and removal of the aqueous phase, (iii) addition of distilled water and removal of the aqueous phase.

Dry the organic phase over sodium sulphate then filter and concentrate to dryness.

A thick, light brown oil is obtained, which is dissolved in 350 ml of a water/trifluoroacetic acid mixture (at 11.10⁻³ mol/litre) and then left at room temperature for 1 h. Concentrate the mixture then absorb five times with 100 ml toluene. Purify the residue on silica gel.

12 g of compound is obtained in the form of a yellow powder.

¹³C NMR (DMSO) 200 MHz δ (ppm): 60.76; 63.82; 92.10; 92.24; 96.75; 96.86.

The ¹³C NMR spectrum (DMSO) 200 MHz corresponds to the expected structure.

### EXAMPLE 3

### Preparation of the Glucose Ester of Stearic Acid (Mostly Ester at Position 3)

Place 0.5 g (1.9 mmol) of diacetone-D-glucose dissolved in 6 ml of dichloromethane, and 0.5 ml (6.1 mmol) of pyridine, in a 50-ml three-necked flask equipped with a condenser and a dropping funnel, under a nitrogen atmosphere.

Maintain the temperature at about 10° C. with an ice water bath.

Add, dropwise, 0.8 ml (2.3 mmol) of the chloride of stearic acid (commercial) in 3 ml of dichloromethane obtained previously, while maintaining the temperature at about 10° C. Then stir the reaction medium for 2 hours at room temperature.

Dilute the pasty mixture obtained by adding 50 ml of dichloromethane. Then wash several times: (i) addition of distilled water and removal of the upper, aqueous solution, (ii) addition of a solution of 1N hydrochloric acid and removal of the aqueous phase, (iii) addition of distilled water and removal of the aqueous phase.

Dry the organic phase over sodium sulphate then filter and concentrate to dryness.

A thick, light brown oil is obtained, which is dissolved in a water/trifluoroacetic acid mixture (⅛) and left at room temperature for 30 minutes. Concentrate the mixture then absorb five times with 100 ml toluene. The residue is recrystallized from MeOH.

0.57 mg of compound is obtained in the form of a yellow powder. The overall yield is 66%.

The ¹H and ¹³C NMR spectra (DMSO) 200 MHz correspond to the expected structure.

### EXAMPLE 4

### Preparation of 3-O-octadeca-9,12-dienoyl-D-glucopyranose

Place 29.6 g of diacetone-D-glucose dissolved in 200 ml of dichloromethane, and 26 ml of triethylamine, in a 500-ml three-necked flask equipped with a condenser and a dropping funnel, under a nitrogen atmosphere.

Maintain the temperature at about 10° C. with an ice water bath.

Add, dropwise, 200 ml of chloride of octadeca-9,12-dienoic (linoleic) acid, while maintaining the temperature at about 10° C. Then stir the reaction medium for 2 hours at room temperature.

Dilute the pasty mixture obtained by adding 200 ml of dichloromethane. Then wash several times:

(i) addition of distilled water and removal of the upper, aqueous solution,

(ii) addition of a solution of 1N hydrochloric acid and removal of the aqueous phase,

(iii) addition of distilled water and removal of the aqueous phase.

Dry the organic phase over sodium sulphate then filter and concentrate to dryness.

21 g of a thick, light brown oil is obtained, which is dissolved in 350 ml of a water/trifluoroacetic acid mixture (at 11.10⁻³ mol/litre) and then left at room temperature for 1 h. Concentrate the mixture then absorb 5 times with 100 ml toluene. Purify the residue on silica gel.

10.8 g of compound is obtained in the form of a yellow oil (yield 64%).

The ¹H and ¹³C NMR spectra (DMSO) correspond to the expected structure.

### EXAMPLE 5

### Activity of the Glucose Ester at 6 of Linoleic Acid with Respect to Sebum Production

The test compounds were evaluated on a model of human sebocytes immortalized in culture, derived from the SZ95 line described in Zouboulis, C. C. et al., Establishment and Characterization of an Immortalized Human Sebaceous Gland Cell Line, J. Invest. Dermatol., 113, 1011-1020 (1999).

The following products were tested:

the D-glucose ester at position 6 of vitamin F prepared according to example 1,

DHEA (dehydroepiandrosterone) marketed by the company SIGMA,

the D-glucose ester at position 3 of stearic acid prepared according to example 3,

the D-glucose ester at position 3 of vitamin F prepared according to example 2, and

the amide at position 2 of glucosamine of linoleic acid prepared by condensation of linoleyl chloride on glucosamine.

The test consists of measuring the quantity of lipids produced by the sebocytes of the cell line (at confluence), with or without active agents present, diluted in DMSO, in such a way that the final amount of DMSO in the basal medium is 0.1%. After 2 days of treatment, the adhering cells are treated with Nile Red (1 μg/ml). The lipids content is then quantified by measuring the fluorescence of the stain (two excitation/emission pairs: 485-540 nm for neutral lipids and 540-620 nm for non-neutral lipids). The results are given for total lipids (combining both measurements).

The test is carried out in decaplicate (assayed products and control) in a 96-well plate and renewed 3 times.

The results obtained are shown in Table I. This also shows the results obtained in the presence of DHEA, a known activator of sebaceous function.

TABLE I

| Product | Variation, lipid/CONTROL |
|---|---|
| (100 μM) | (%) |
| DHEA | +80 |
| Glucose ester at 6 of vitamin F | +583 |
| Glucose ester at 3 of stearic acid | +100 |
| Glucose ester at 3 of vitamin F | +74 |
| Amide at 2 of the glucosamine of linoleic | +40 |
| acid | |

As can be seen from this table, all of the compounds according to the invention cause an increase in sebocyte lipogenesis. This increase is particularly significant for the glucose ester at 6 of vitamin F and the glucose ester at 3 of stearic acid—these compounds give rise to an increase that is greater than that observed with DHEA at the same dose.

### EXAMPLE 6

### Determination of the Cytotoxicity of the Glucose Ester at 6 of Linoleic Acid

The tolerance of the glucose ester at 6 of linoleic acid was determined by measuring the cytotoxicity of the product on SZ 95 sebocytes, with linoleic acid alone as control.

The test conditions are identical to those examined in example 1. Cytotoxicity is measured by the production of LDH in the basal medium, according to the method described in Thomas J P et al.; Lethal damage to endothelial cells by oxidized low density lipoprotein: role of selenoperoxidases in cytoprotection against lipid hydroperoxide and iron mediated reactions. Journal of lipid research 34: 479-490. 1993.

The results are presented in Table II.

TABLE II

| Product | Variation, LDH/control |
|---|---|
| (100 μM) | (%) |
| Linoleic acid | +20 |
| Glucose ester at 6 of linoleic acid | Not significant |

No cytotoxicity was found with the glucose ester at 6 of linoleic acid with respect to sebocytes.

### EXAMPLE 7

### Comparative Study of the Peroxidizability in the Air of Compounds of the Invention Relative to Compounds not Corresponding to the Invention

The purpose of this study is to evaluate the peroxidizability of various molecules or mixtures of molecules by carrying out various tests. These tests consist of measuring the proportion of molecules still intact after storage for two months, in air, at room temperature (about 20 to 25° C.). The loss of the starting product is monitored by HPLC with UV detection (210 nm).

The following products were tested:

1. linoleic acid: octadeca-9,12-dienoic acid marketed by the company Aldrich,

2. methyl linoleate: marketed by the company Aldrich under the reference 10,335-7,

3. vitamin F (containing 75 to 80% of linoleic acid) marketed by the company Stéarinerie Dubois under the reference 14043,

4. monoester of linoleic acid and of D-glucose at position 6: 6-O-octadeca-9,12-dienoyl-D-glucopyranose: prepared according to the method described in patent EP485251,

5. glucose ester of vitamin F (mostly ester at position 6): mixture obtained in example 1.

Results

In the conditions described above, products 4 and 5 have 30% of intact molecules.

In the same conditions, products 1, 2 and 3 no longer contain intact molecules.

These results show that the ester of linoleic acid and of D-glucose at position 6, and the ester of vitamin F and of D-glucose, mostly at position 6, have better stability with respect to oxidation in the air than native linoleic acid or vitamin F. These particular products also have better stability with respect to oxidation in the air than other esters, especially the methyl ester.

### EXAMPLE 8

### Investigation of the Stability of Compounds of the Invention

The stability of the compounds according to the invention was evaluated (measurement of hydrolysis of the esters).

Solutions were prepared at 0.1 wt. % of the compounds in ethanol/isopropanol/water mixture (64/16/20 by volume). These solutions were left in a thermostat at 45° C., for 2 months.

Then the percentage hydrolysis of glucopyranose linoleate was determined by HPLC.

The results are shown in Table III.

TABLE III

| Compound | % hydrolysis |
|---|---|
| Glucose ester of vitamin F (mostly ester at position 6) | 3 |
| mixture obtained in example 1 | |
| 6-O-octadeca-9,12-dienoyl-D-glucopyranose | 7 |
| Glucose ester of vitamin F (mostly at position 3) | 17 |
| mixture obtained in example 2 | |
| 3-O-octadeca-9,12-dienoyl-D-glucopyranose obtained | 30 |
| in example 4 | |

The compounds of the invention therefore exhibit a percentage hydrolysis of the glucopyranose linoleate less than or equal to 30% in the test conditions. They therefore represent different forms in which the glucopyranose linoleate possesses good stability.

### EXAMPLE 9

### Measurement of the Synthesis of 13-HODE by Hair Follicles Surviving in Culture

300 hair follicles, from a sample obtained from a volunteer donor, were dissected by the technique described in patent application FR 2 736 721. Then the hair follicles were placed in a complete basal medium marketed under the name “William's E” by the company Gibco. After surviving for 16 hours in vitro (apparent viability established under a binocular magnifier), batches of 25 hair follicles were selected. Each batch of 25 hair follicles was then placed in 500 μl of William's E medium in a stove at 37° C. under 5% of carbon dioxide.

At t=0, either a control solution (dimethyl sulphoxide at a final concentration of 0.2%), or a solution of linoleic acid (50 mM) in dimethyl sulphoxide at a final concentration of linoleic acid of 10 μM, or a solution of a glucose ester and of vitamin F as prepared in example 1 (50 mM) in dimethyl sulphoxide at a final concentration of glucose ester and of vitamin F of 10 μM was introduced into the basal medium of each batch. The various batches were incubated at 37° C. under 5% carbon dioxide. Samples (50 μl) were taken after thirty minutes, one hour and two hours of incubation.

Determination of 13-HODE

The assay was carried out using the immuno-enzymatic kit marketed under reference “EA81” by the company Oxford Biomedical Research. Each sample was placed in 150 μl of the dilution buffer supplied in the kit (which corresponds to dilution at ¼). The assay protocol specified by the manufacturer is then followed.

The results, presented as a histogram in FIG. 1, are expressed in picograms of 13-HODE for 25 hairs that survived.

In the histogram in FIG. 1, the light grey represents the concentration of 13-HODE measured in the control conditions; the dark grey represents the concentration of 13-HODE measured when the ester of vitamin F and of glucose (mostly at position 6) was added at t=0 to the final concentration of 10 μM and the white represents the concentration of 13-HODE measured when linoleic acid was added at t=0 to the final concentration of 10 μM.

The results obtained show that bringing hair follicles into contact with an ester of vitamin F and of glucose, mostly at position 6, according to the present invention, leads to a considerable increase in the concentration of 13-HODE in the incubation medium.

It can also be seen that the concentration of 13-HODE measured after bringing hair follicles into contact with the ester of vitamin F and of glucose mostly at position 6 according to the invention is markedly higher, not only than that observed when the hair follicles are brought into contact with the control solution, but also than that observed when the hair follicles are brought into contact with the natural precursor of 13-HODE, namely linoleic acid.

### EXAMPLE 10

### Cosmetic and Dermatologic Compositions According to the Invention

These compositions are prepared in a manner familiar to a person skilled in the art. The quantities shown in these examples are percentages by weight.

| A. | Lotion | |
|---|---|---|
| | Compound of example 1 | 1% |
| | Salicylic acid | 1% |
| | Propyleneglycol | 5% |
| | Alcohol | 87% |
| | Water qsf | 100% |

This lotion can be used in the evening for reviving sebaceous function and/or for improving the condition of the skin barrier.

| B. | Emollient cream | |
|---|---|---|
| | Compound of example 1 | 1% |
| | n-Octanoyl-5-salicylic acid | 1% |
| | Methylparaben ® | 0.1% |
| | Propylparaben ® | 0.1% |
| | Lanolin | 5% |
| | Vaseline oil | 4% |
| | Sesame oil | 4% |
| | Cetyl alcohol | 5% |
| | Glycerol monostearate | 2% |
| | Triethanolamine | 1% |
| | Propyleneglycol | 5% |
| | Carbomer 940 ® marketed by | 0.1% |
| | the company NOVEON | |
| | Water qsf | 100% |

| C. | Anti-inflammatory ointment. | |
|---|---|---|
| | Compound of example 1 | 2% |
| | Hydrocortisone | 1% |
| | Glycerol monostearate | 3% |
| | Propyleneglycol | 12% |
| | Petrolatum | 81.9% |
| | Water qsf | 100% |

| D. | Gel | |
|---|---|---|
| | Compound of example 1 | 1% |
| | Salicylic acid | 1% |
| | Hydroxypropyl cellulose | 1% |
| | PPG-12-Buteth-16 ® marketed by | 2% |
| | the company AMERCHOL | |
| | Triethanolamine | 0.2% |
| | Propyleneglycol | 5% |
| | Alcohol | 45% |
| | Carbomer 940 ® marketed by | 0.2% |
| | the company NOVEON | |
| | Water qsf | 100% |

| E. | Anti-aging cosmetic cream | |
|---|---|---|
| | Compound of example 1 | 3% |
| | Lyophilized extract of rosemary | 0.2% |
| | Glycerol stearate | 2% |
| | Polysorbate 60 ® marketed by | 1% |
| | the company UNIQEMA | |
| | Stearic acid | 1.4% |
| | Triethanolamine | 0.7% |
| | Carbomer ® marketed by the | 0.4% |
| | company NOVEON | |
| | Olive oil | 12% |
| | Liquid fraction from shea butter | 12% |
| | Octyldodecanol | 6% |
| | Isononyl isononanoate | 10% |
| | Antioxidant | 0.05% |
| | Perfume | 0.5% |
| | Preservatives | 0.3% |
| | Water qsf | 100% |

| F. | Pharmaceutical anti-aging cream | |
|---|---|---|
| | Compound of example 1 | 2% |
| | Retinoic acid | 0.025% |
| | Glycerol | 3% |
| | Xanthan gum | 0.1% |
| | Oxyethylenated sorbitan stearate | 0.9% |
| | Mixture of PEG-100 stearate and | 2.1% |
| | glyceryl stearate ® marketed by | |
| | the company INOLEX | |
| | Cetyl alcohol | 2.6% |
| | Isononyl isononanoate | 11% |
| | Octyldodecanol | 15% |
| | Butylhydroxytoluene | 0.1% |
| | Octocrylene | 0.1% |
| | Triethanolamine | 2% |
| | Tocopherol acetate | 1% |
| | Preservatives | 0.6% |
| | Water qsf | 100% |

| G. | Moisturizing cream | |
|---|---|---|
| | Compound of example 1 | 3% |
| | Triethanolamine | 0.3% |
| | Mixture of PEG-100 stearate and | 2.5% |
| | glyceryl stearate ® marketed by | |
| | the company INOLEX | |
| | PEG-50 stearate | 2.5% |
| | Cetyl alcohol | 1% |
| | Stearyl alcohol | 3% |
| | Isononyl isononanoate | 20% |
| | Propylparaben ® | 0.1% |
| | Carbopol ® marketed by the | 0.3% |
| | company NOVEON | |
| | Water qsf | 100% |

1. Use of at least one amide, sugar mono- or polyester of fatty acid, as active principle, for the preparation of a cosmetic or pharmaceutical composition intended for preventing and/or treating dry skin.

2. Use of at least one amide, sugar mono- or polyester of fatty acid, as active principle, for the preparation of a cosmetic or pharmaceutical composition intended for treating oligoseborrhoeic dry skin.

3. Use of at least one amide, sugar mono- or polyester of fatty acid, as active principle, for the preparation of a cosmetic or pharmaceutical composition intended for stimulating sebum production.

4. Use according to claim 1, characterized in that the fatty acid has more than 14 carbon atoms.

5. Use according to claim 1, characterized in that the fatty acid can be saturated or can contain one or more double bonds.

6. Use according to claim 1, characterized in that the fatty acid is selected from palmitic, stearic, arachidic, behenic, lignoceric, palmitoleic, oleic, linoleic, and linolenic acids, notably in their α or γ forms and arachidonic acid.

7. Use according to claim 1, characterized in that the fatty acid is linoleic acid or stearic acid.

8. Use according to claim 1, characterized in that the fatty acid is linoleic acid.

9. Use of at least one amide, sugar mono- or polyester of linoleic acid, as active principle, for the preparation of a cosmetic or pharmaceutical composition intended for generating 13-hydroxyoctadecadienoic acid in the cutaneous epidermis.

10. Use of at least one amide, sugar mono- or polyester of linoleic acid, as active principle, for the preparation of a cosmetic or pharmaceutical composition intended for treating and/or preventing skin disorders and/or disorders of the pilosebaceous unit associated with linoleic acid deficiency.

11. Use according to claim 1, characterized in that the sugar is a mono- or oligosaccharide.

12. Use according to claim 11, characterized in that the sugar is a mono- or disaccharide.

13. Use according to claim 1, characterized in that the sugar is an isomer from the D series of mono- and disaccharides.

14. Use according to claim 1, characterized in that the sugar is or is derived from at least one pentose and/or hexose.

15. Use according to claim 1, characterized in that the sugar can be in its α- and/or β-cyclized form.

16. Use according to claim 1, characterized in that the sugar is a mono- or disaccharide selected from talose, fucose, ribose, idose, arabinose, gulose, xylose, lyxose, altrose, allose, glucose, mannose, galactose, lactose, sucrose, trehalose, cellobiose, maltose, fucose alpha 1-3 glucose, fucose alpha 1-4 glucosamine, fructose, glucosamine, fructosamine and galactosamine and their derivatives.

17. Use according to claim 1, characterized in that the sugar is a monosaccharide from the pentose series.

18. Use according to claim 17, characterized in that it is selected from lyxose, xylose, arabinose and ribose.

19. Use according to claim 1, characterized in that the sugar is a monosaccharide from the hexose series.

20. Use according to claim 19, characterized in that it is selected from talose, fucose, galactose, idose, gulose, mannose, glucose, altrose, allose, glucosamine, galactosamine, N-acetyl glucosamine, N-acetyl galactosamine and fructose.

21. Use according to claim 16, characterized in that the sugar is a disaccharide selected from maltose, sucrose, cellobiose, trehalose, lactose, fucose alpha 1-3 glucose and fucose alpha 1-4 glucosamine.

22. Use according to claim 1, characterized in that the sugar is the αD- or βD-isomer of glucose.

23. Use according to claim 1, characterized in that the sugar is mono- or polyesterified by the said fatty acid.

24. Use according to claim 1, characterized in that the sugar is a mono- or disaccharide esterified at position 1, 2, 3, 4 and/or 6.

25. Use according to claim 1, characterized in that the sugar is a mono- or disaccharide esterified at position 1, 2, 3 and/or 6.

26. Use according to claim 1, characterized in that the said composition contains at least the glucose monoester at position 1, 3 or 6 of linoleic acid.

27. Use according to claim 26, characterized in that it is the ester at position 6 of αD- or of βD-glucose of linoleic acid.

28. Use according to claim 26, characterized in that the said ester is used in the form of a mixture with at least one glucose ester of another fatty acid.

29. Use according to claim 28, characterized in that the other fatty acid is stearic acid and/or oleic acid.

30. Use according to claim 28, characterized in that the proportion by weight of ester of linoleic acid and of glucose relative to the total weight of the said mixture is from 40 to 90%, notably it is greater than or equal to 50%, in particular greater than or equal to 60%, more particularly less than or equal to 80%, notably less than 75% and in particular varies from 68 to 72%.

31. Use according to claim 28. characterized in that the said mixture contains at least one ester of stearic acid and of glucose in a proportion by weight relative to the total weight of the said mixture from 0.1% to 7%, notably greater than or equal to 0.5%, in particular greater than or equal to 1%, notably less than or equal to 5%, and in particular in a proportion varying from 2 to 4 wt. %.

32. Use according to claim 28, characterized in that the said mixture contains at least one ester of oleic acid and of glucose in a proportion by weight relative to the total weight of the said mixture from 5 to 20%, notably greater than or equal to 8%, in particular greater than or equal to 10%, more particularly greater than or equal to 12%, and in particular less than or equal to 17% and notably in a proportion varying from 14 to 15 wt. %.

33. Use according to claim 28, characterized in that the said mixture contains at least one ester of palmitic acid and of glucose in a proportion by weight relative to the total weight of the said mixture from 2 to 20%, notably greater than or equal to 5%, in particular greater than or equal to 7%, and notably less than or equal to 15% and in particular in a proportion varying from 9 to 12 wt. %.

34. Use according to claim 28, characterized in that the said mixture contains at least one ester of fatty acid and of glucose, the said acid being selected from lauric, myristic, arachidic, behenic, lauroleic, myristoleic, palmitoleic and linolenic acids, in a proportion by weight relative to the total weight of the said mixture less than or equal to 10%, notably varying from 0.1 to 4%, and in particular from 0.15 to 2%.

35. Use according to claim 28, characterized in that the said esters are monoesters.

36. Use according to claim 35, characterized in that the said mixture contains additionally at least one diester of glucose and of a fatty acid or of two different fatty acids selected from linoleic, oleic, palmitic, stearic, lauric, myristic, arachidic, behenic, lauroleic, myristoleic, palmitoleic and linolenic acids, in a proportion by weight relative to the total weight of the said mixture less than or equal to 10%, notably from 0.1 to 4%, and in particular from 0.15 to 2%.

37. Use according to claim 28, characterized in that the said mixture contains:
from 40 to 80 wt. %, preferably 60 to 75 wt. %, preferentially 68-72 wt. %, of monoester of glucose and of linoleic acid,
from 10 to 20 wt. %, preferably 12 to 17 wt. %, preferentially 14-15 wt. %, of monoester of glucose and of oleic acid,
from 5 to 20 wt. %, preferably 7 to 15 wt. %, preferentially 9-12 wt. %, of monoester of glucose and of palmitic acid,
from 0.5 to 7 wt. %, preferably 1 to 5 wt. %, preferentially 2-4 wt. %, of monoester of glucose and of stearic acid,
from 0 to 10 wt. %, notably 0.10-4 wt. %, or even 0.15-2 wt. %, of one or more monoesters of glucose and of lauric, myristic, arachidic, behenic, lauroleic, myristoleic, palmitoleic and/or linolenic acid,
from 0 to 10 wt. %, notably 0.10-4 wt. %, or even 0.15-2 wt. %, of diesters of glucose and of one or more acids selected from lauric, myristic, arachidic, behenic, lauroleic, myristoleic, palmitoleic, linoleic, oleic, palmitic, stearic and/or linolenic acids.

38. Use according to claim 37, characterized in that the said mixture contains:
from 40 to 80 wt. %, preferably 60 to 75 wt. %, preferentially 68-72 wt. %, of ester of glucose and of linoleic acid and principally 6-O-octadeca-9,12-dienoyl-D-glucopyranose, 1-O-octadeca-9,12-dienoyl-D-glucopyranose, 2-O-octadeca-9,12-dienoyl-D-glucopyranose and/or 3-O-octadeca-9,12-dienoyl-D-glucopyranose,
from 10 to 20 wt. %, preferably 12 to 17 wt. %, preferentially 14-15 wt. %, of ester of glucose and of oleic acid, and principally 6-O-octadeca-9-enoyl-D-glucopyranose, 3-O-octadeca-9-enoyl-D-glucopyranose, 1-O-octadeca-9-enoyl-D-glucopyranose and/or 2-O-octadeca-9-enoyl-D-glucopyranose,
from 5 to 20 wt. %, preferably 7 to 15 wt. %, preferentially 9-12 wt. %, of ester of glucose and of palmitic acid, and principally 6-O-hexadecanoyl-D-glucopyranose, 3-O-hexadecanoyl-D-glucopyranose, 1-O-hexadecanoyl-D-glucopyranose and/or 2-O-hexadecanoyl-D-glucopyranose,
from 0.5 to 7 wt. %, preferably 1 to 5 wt. %, preferentially 2-4 wt. %, of ester of glucose and of stearic acid and principally 6-O-octadecanoyl-D-glucopyranose, 3-O-octadecanoyl-D-glucopyranose, 1-O-octadecanoyl-D-glucopyranose and/or 2-O-octadecanoyl-D-glucopyranose,
from 0 to 10 wt. %, notably 0.10-4 wt. %, or even 0.15-2 wt. %, of one or more esters of glucose and of lauric, myristic, arachidic, behenic, lauroleic, myristoleic, palmitoleic and/or linolenic acid,
from 0 to 10 wt. %, notably 0.10-4 wt. %, or even 0.15-2 wt. %, of diesters of glucose and of one or more acids selected from lauric, myristic, arachidic, behenic, lauroleic, myristoleic, palmitoleic, linoleic, oleic, palmitic, stearic and/or linolenic acids.

39. Use according to claim 28, characterized in that the said mixture can be obtained by esterification of D-glucose by vitamin F.

40. Use according to claim 1, characterized in that the said composition contains the said active principle in proportions varying from 0.001 to 30 wt. %, and in particular from 0.01 to 15 wt. %, notably from 0.1 to 5 wt. % relative to the total weight of the composition.

41. Use according to claim 1, characterized in that the said composition additionally contains an effective quantity of at least one other active agent.

42. Use according to claim 41, characterized in that the said agent is selected from moisturizing agents, agents that activate the sebaceous glands, agents that stimulate proliferation of keratinocytes, agents that stimulate differentiation of keratinocytes, anti-inflammatory agents, calmatives, antibacterial agents, calcium antagonists, free radical trapping agents, and filters that are active in UV-A and/or UV-B.

43. Use according to claim 1, characterized in that the composition is applied topically.

44. Method of cosmetic treatment of the skin, characterized in that at least one composition as defined in claim 1 is applied on the area to be treated.
